(19) United States
(12) Patent Application Publication
(10) Pub. No.: US 2009/0104292 A1
(43) Pub. Date: Apr. 23, 2009

(54) TOPICAL ARNICA TREATMENT FOR REDUCING BRUISING

(75) Inventor: Murad Alam, Chicago, IL (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: SCAR CONSULTANTS, LLC, Chicago, IL (US)

(21) Appl. No.: 12/255,974

(22) Filed: Oct. 22, 2008

Related U.S. Application Data
(60) Provisional application No. 60/981,864, filed on Oct. 23, 2007.

(51) Int. Cl. A61K 36/00

(52) U.S. Cl. 424/725

(57) ABSTRACT

A method of preventing post-surgery, post-laser treatment or post-traumatic bruising of the skin comprising applying topically to the affected area of the skin a treatment comprising at least 15% by weight Arnica Montana and preferably 30-40% by weight Arnica Montana.

### CROSS-REFERENCE TO RELATED PATENT APPLICATIONS

This patent application claims the benefit of U.S. Provisional Patent Application No. 60/981,864, filed Oct. 23, 2007.

### FIELD OF THE INVENTION

This invention relates to methods for improving wound healing and, more particularly, to topically applied treatments for preventing post-surgery, post-laser treatment or post-traumatic bruising of the skin.

### BACKGROUND OF THE INVENTION

Surgery, laser-treatments, and trauma often cause bruising. Once a bruise has developed because of a medical procedure or trauma, the bruise will take some time to resolve and may be unsightly until then, particularly if it occurs on the face or other visible areas. Also, blood thinners and anticoagulation agents that are present in a patient's system can aggravate such bruise formation.

Current methods for minimizing bruising following surgery or laser treatment entail discontinuing ingestion of blood thinners and anticoagulation agents (both prescription and nonprescription products) prior to performing such procedures. Unfortunately, it is not always possible to ensure that such agents that can cause bruising are discontinued. Also, some prescribed anticoagulants may be necessary to protect against blood clots. Indeed, it is often medically unsafe to stop medications that may cause bruising as cessation of prescription medications can induce a hypercoaguable state and predispose to stroke, pulmonary embolus, or heat attack in susceptible patients.

Physicians and hospitals provide patients lists of medications to discontinue before procedures, and they remind patients to stop these and start a bruising-reducing medication before procedures. Compliance with stopping medications before treatment nevertheless is poor and wastes a great deal of manpower. Also, even if instructed to do so, patients may forget to stop taking anticoagulants prior to surgery or they may refuse to stop taking such medications. This is particularly the case as to herbal medications like Gingko biloba, or supplements like Vitamin E, that can cause or aggravate bruising. Another problem with cessation of blood thinners and anticoagulation agents prior to the medical procedure is that if such bruising does not in fact occur, the pretreatment regimen would be a needless expenditure of time and effort.

Using medications before the procedure to prevent bruising can be unrealistic because not all procedures are preplanned, and patients routinely forget to take medications ahead of time. Additionally, some patients may not wish to take additional oral medications to prevent bruising, or this may be medically contraindicated. Finally, if bruising is secondary to trauma, there is no way of predicting this bruising, and hence no way of avoiding or ameliorating it by pre-medicating or stopping medications ahead of time.

### BRIEF SUMMARY OF THE INVENTION

The present invention entails the application of high levels of Arnica Montana to areas of the skin subjected to surgery, laser treatment or other trauma after they develop bruising. Preferably, the Arnica Montana will be applied in the form of an ointment. It may also be applied in the form of aqueous solutions, creams, gels, moisturizers, topical cosmetics and cosmeceutical products. Topical treatment with high levels of Arnica Montana requires no cessation of other medications and need only be implemented after bruises develop. The ointment may be delivered in a petrolatum base. In this form it also provides soothing moisturization to the area of the bruise.

Arnica Montana has been used in the past at low levels (under 10% by weight) as one ingredient among many others in homeopathic and other formulations applied topically or ingested and recommended for a range of different purposes ranging from stiffness, pain, and bruising to fatigue, rheumatism and migraine headaches. However, it has never been previously suggested to apply Arnica Montana as a topical medicament for bruises at the high application levels of this invention. The prior lower levels of Arnica when applied topically or taken orally are ineffective in resolving bruising.

The concentration of the Arnica Montana in the ointment should be at least 15% by weight on a weight/weight basis, based on the weight of the carrier and any secondary ingredients. Preferably, the level of Arnica Montana should be at least 20% by weight and more preferably, the Arnica Montana should be at a concentration up to the limit that can be achieved in the carrier. The Arnica Montana may be, e.g., in a formulation comprising Arnica 20% v/v in Pluronic organogel, Arnica tincture lecithin isopropyl palmitate solution, Pluronic gel 30% and Krisgel up to 3% if needed to enhance gel structure, with the balance white petrolatum USP. However, concentrations of up to about 30% by weight in a transdermal gel and up to 40% by weight in an aqueous solution are currently preferred.

In formulations as described above, the organogel acts as a diluent and gelling agent. Other gelling agents that may be used, include but are not limited to, cellulose ethers, alginates, polyacrylates (carboners), carboxyvinyl polymer, bentonite, gelatin, tragcanth, polyvinylpyrrolidone, polyvinyl alcohol, and polyoxyethylene/polyoxypropylene block copolymers. Pluronic organogels are available from BASF of Parsippany, N.J. The Krisgel also acts as a diluent and is available from PCCA of Houston, Tex. Finally, it should be noted that the treatment may be formulated with any suitable diluents, gelling agents, and other secondary ingredients, as recognized by those skilled in the art, so long as the ingredients are compatible with and do not interfere with the activity of the Arnica Montana.

A successful randomized clinical trial showed that an ointment of 20% by weight of Arnica Montana in petrolatum base applied sparingly twice a day for two weeks to a laser-induced bruise about 1 cm in diameter speeded clinical resolution of bruising and both a 30% by weight transdermal gel and a 40% by volume aqueous solution produced a yet more accelerated resolution of bruising, with a reduction of bruising to minimal of 9 days for treatment versus 14 days for a placebo. (The 40% by volume gel is believed to be substantially equivalent to about 40% by weight). In all cases, approximately 5-15 grams of the ointment were used to treat a small (1 cm diameter) bruise for 1-2 weeks. These trials established that superior results were obtained as compared to treatment with Vitamin K, Vitamin K & Retinol, and Hirudin.

Thus, topical application of an ointment containing Arnica Montana at levels of at least 15%, preferably at least 20% by weight, and more preferably about 30% by weight as a gel or about 40% by weight as an aqueous solution (preferably by twice daily applications) to the site of any bruise that develops speeds the resolution of bruises. It is also preferred that the ointment be applied at a level of about 1 gram per 10 sq. cm per application. Additionally, the effectiveness is improved under occlusion of tape, plastic wrap, etc. Unlike oral medications, the treatment does not need to be taken by mouth, cannot interact with other oral medications, and does not need to be started before a bruise develops in order to work. Also, the practice of this invention does not entail stopping medically necessary medications or stopping nutritional supplements.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What we claim:

1. A method of preventing post-surgery, post-laser treatment or post-traumatic bruising of the skin comprising:
applying topically to the affected area of the skin a treatment comprising at least 15% by weight Arnica Montana.

2. The method of claim 1 in which the treatment comprises at least 20% by weight Arnica Montana.

3. The method of claim 1 in which the treatment comprises at least 30% by weight Arnica Montana.

4. The method of claim 1 in which the treatment comprises a transdermal gel.

5. The method of claim 3 in which the treatment comprises a transdermal gel.

6. The method of claim 1 in which the treatment comprises an aqueous solution at least 40% by weight Arnica Montana.

7. The method of claim 1 in which the treatment is delivered as an ointment in a petrolatum base.

8. A treatment for preventing post-surgery, post-laser treatment or post-traumatic bruising of the skin comprising:
an ointment or a solution comprising at least 15% by weight Arnica Montana.

9. The treatment of claim 8 in which the Arnica Montana is present at a level of at least 20% by weight Arnica Montana.

10. The treatment of claim 8 in which the Arnica Montana is present at a level of at least 30% by weight Arnica Montana.

11. The treatment of claim 8 in which the Arnica Montana is present at a level of at least 40% by weight Arnica Montana.

12. The treatment of claim 8 in which the Arnica Montana is in a form chosen from the group consisting of aqueous solutions, creams, gels, moisturizers, topical cosmetics and cosmeceutical products.

13. The treatment of claim 8 including diluents and gelling agents.

14. The treatment of claim 13 in which the diluents and gelling agents are chosen from the group consisting of organogels, cellulose ethers, alginates, polyacrylates, carboxyvinyl polymer, bentonite, gelatin, tragcanth, polyvinylpyrrolidone, polyvinyl alcohol, and polyoxyethylene/polyoxypropylene block copolymers.
